(19) United States
(12) Patent Application Publication
(10) Pub. No.: US 2025/0157199 A1
(43) Pub. Date: May 15, 2025

(54) DE-IDENTIFICATION OF FACIAL IMAGES

(71) Applicant: Atapir, Inc., San Rafael, CA (US)

(72) Inventors: Arthur Wallace, San Rafael, CA (US); Kai Kuck, Park City, UT (US)

(21) Appl. No.: 18/943,555

(22) Filed: Nov. 11, 2024

Related U.S. Application Data
(60) Provisional application No. 63/598,023, filed on Nov. 10, 2023.

(51) Int. Cl. G06V 10/774; G06V 10/82; G16H 10/60; G16H 50/20

(52) U.S. Cl. CPC G06V 10/7753; G06V 10/82; G16H 10/60; G06V 2201/03; G16H 50/20

(57) ABSTRACT

A method or system uses de-identified images collected from patients in association with de-identified data collected as part of medical care to train machine learning, machine vision, deep learning, or other algorithms that associate an outcome variable with an input image or video image(s).

### CLAIM OF PRIORITY

This application claims priority to U.S. Provisional Application Ser. No. 63/598,023, filed on Nov. 10, 2023, the entire contents of which are hereby incorporated by reference.

### GOVERNMENT SUPPORT STATEMENT

The technology described here was made without any support from any government.

### TECHNICAL FIELD

This document describes data processing and analysis technology for de-identifying facial images, e.g., to enable unsupervised machine learning using de-identified PHI data.

### BACKGROUND

Artificial intelligence, deep learning, and machine vision algorithms require large databases of information to train neural and deep learning networks and other algorithms. Artificial intelligence systems for health care require access to PHI (“PHI”) to train networks. Access to PHI requires patient consent which can be time consuming, expensive, and complex to obtain precluding the creation of large databases necessary for training artificial intelligence algorithms.

### SUMMARY

This document describes technology designed, among other things, to de-identify facial images to enable unsupervised machine learning using de-identified PHI data without the need for obtaining patient consent.

In one aspect a method or system to de-identify facial images may include using de-identified images collected from patients in association with de-identified data collected as part of medical care to train machine learning, machine vision, deep learning, or other algorithms that associate an outcome variable with an input image or video image(s).

In another aspect, a method or system may store de-identified images or video images for use in machine vision, machine learning, neural network, deep learning, or other algorithms that associate an outcome variable with an input image or video image(s).

In another aspect, method or system may use unsupervised learning based on video images and data collected as part of medical care to train machine learning, machine vision, deep learning, or other algorithms that associated an outcome variable with an input image or video image(s).

In another aspect, a method or system may use unsupervised learning based on de-identified video images and de-identified data collected as part of medical care to train machine learning, machine vision, deep learning, or other algorithms that associated an outcome variable with an input image or video image(s).

Other features, aspects and potential advantages will be apparent from the accompanying description and figures.

### DESCRIPTION OF DRAWINGS

FIG. 1 shows a patient in bed with a camera array observing a display of vital sign data.

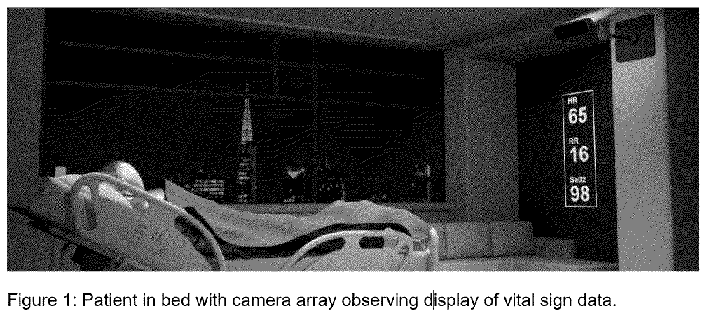

FIG. 2 shows an Audiovisual Detection Monitor (“AVD-M”) prototype with pulse oximeter on finger to check accuracy.

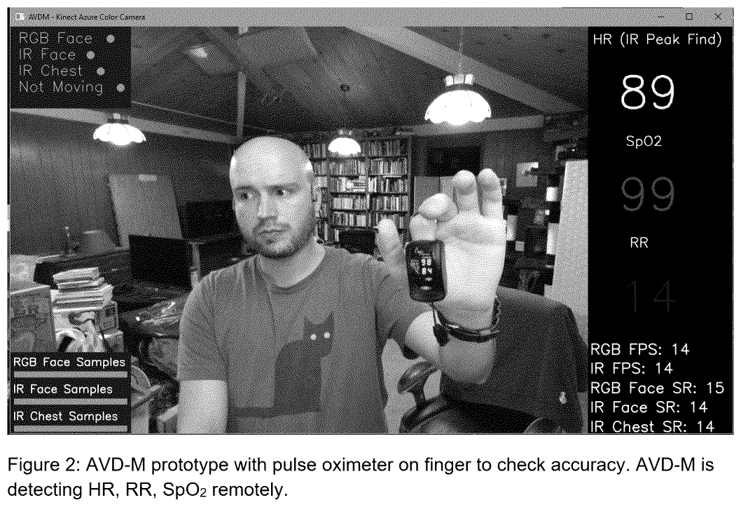

FIG. 3 shows an AVD-M prototype with remote measurement of pulse oximetry, heart rate, and respiratory rate.

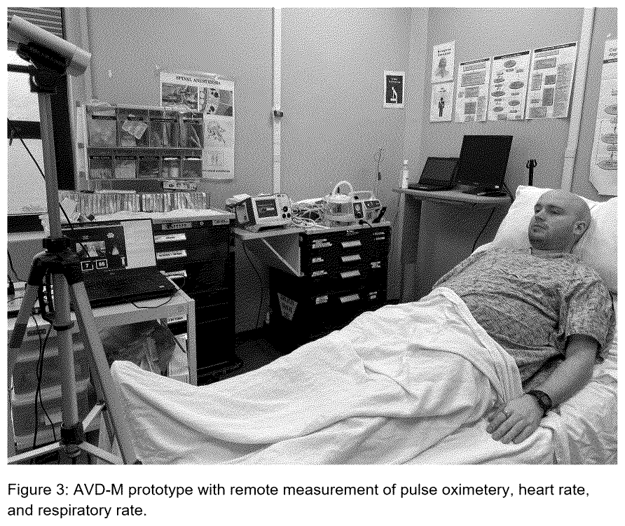

FIG. 4 is a schematic diagram showing an example of a computing device and a mobile computing device.

Like reference symbols in the various drawings indicate like elements

### DETAILED DESCRIPTION

Artificial intelligence, deep learning, and machine vision algorithms require large databases of information to train neural and deep learning networks and other algorithms. Artificial intelligence systems for health care require access to Personal Health Information (“PHI”) to train networks. Access to PHI requires patient consent which can be time consuming, expensive, and complex to obtain precluding the creation of large databases necessary for training artificial intelligence algorithms.

De-identified health information is data that cannot be used to identify a person, or there is no reasonable basis to believe it can be used. De-identification is usually done when sharing patient data from clinical studies or registries to prevent participants from being identified. De-identification can be done by removing specific identifiers, such as names, social security numbers, email addresses, and more. It can also be done by storing personally identifiable information (PII) in a separate, secure database. De-identification allows data to be used for research, policy assessment, comparative effectiveness studies, and other purposes.

The use of de-identified administrative data, which is data that is collected as part of routine health care and processed to remove identification data, does not require patient consent for use, and can dramatically expand the dataset available to train artificial intelligence algorithms. Supervised learning in artificial intelligence is defined by its use of labeled datasets to train algorithms that classify data or predict outcomes accurately. Labeling datasets is labor intensive and precludes the use of very large datasets secondary to the requirement for labeling. Unsupervised machine learning uses algorithms to analyze and cluster unlabeled datasets. Unsupervised learning algorithms discover hidden patterns or data groupings without the need for human intervention or labeling. Supervised machine learning is much more resource-intensive than unsupervised learning because of the need for labeled data. The use of de-identified health or administrative data collected as part of routine clinical care allows the use of unsupervised learning for training healthcare artificial intelligence algorithms with large datasets without the requirement, expense, and complexity for labeling and patient consent.

Machine vision artificial intelligence algorithms that utilize camera-based systems, can image the patient's face and body potentially exposing the patient. Facial images are inherently PHI and identifiable. The ability to de-identify a facial image would make images collected during health care de-identified information and available for analysis for the training of machine vision artificial intelligence algorithms without patient consent. De-identification of facial images, collected as part of routine health care administrative data, combined with de-identified administratively collected hemodynamic, data such as heart rate, blood pressure, pulse oximetry, respiratory rate, would allow the training of artificial intelligence algorithms that utilize machine vision to monitor vital signs, without the requirement for patient consent to utilize the data. The present system describes methods to de-identify facial images so that they can be used for training of machine vision algorithms without subject or patient consent. De-identification of patient data also allows the storage of patient video files to allow machine vision training for rare events such as stroke, shock, falls without patient consent.

Machine vision systems require large data sets for training. Healthcare data is protected by the Health Insurance Portability and Accountability Act (HIPAA). Patient consent takes time and significant expense precluding the creation of large data sets essential for training of machine vision systems. There are multiple approaches to machine vision training including supervised learning, where data are labeled for the target parameter being trained on, and unsupervised learning, where unlabeled data is used for training. In medical care, significant amounts of data are collected as part of routine clinical care and monitoring. Vital signs including blood pressure, heart rate, respiratory rate, pulse oximetry (SpO₂) are measured either continuously or intermittently. These vital sign data are stored and recorded but access to them for research or development either requires de-identification or patient consent. De-identified data describes records that have a re-identification code and have sufficient personally identifiable information removed or obscured so that the remaining information does not identify an individual and there is no reasonable basis to believe that the information can be used to identify an individual. De-identification of vital signs data is relatively easy, the patient's name is replaced with a code number and the vital signs are available for use in research or analysis. Video recordings of a patient's face are inherently identifiable, and therefore constitute PHI.

Facial images are easily identifiable as the patient and therefore constitute PHI. Use of facial images requires consent and specific consent for the use of facial images. If facial images are made non-recognizable they become de-identified data and can be used in research or analysis without patient consent. Moreover, video recordings of patients in-hospital are protected. Patients may be in compromising situations and video recordings make that private information problematic. A method to de-identify the face, would allow the recording of video images from the hospital safer to avoid problems with release of PHI and make that video data available for research and analysis without patient consent.

Development of remote, non-contact, patient monitoring systems that utilize machine vision require recording of patients and patient data and specifically facial information. The development of remote, non-contact, machine vision-based monitoring systems designed for patients in hospitals, nursing homes, at home, adults, infants, or children, who may have cardiovascular, respiratory, medical, psychiatric, neurologic, vascular, or other problems. Patients in hospitals and nursing homes commonly are prescribed medications such as opiates and sedatives that suppress respiration. These medications can lead to hypercapnia, hypoventilation, obstructive sleep apnea, respiratory depression, and ultimately, respiratory and or cardiac arrest. The AVD-M monitor is designed to detect respiratory depression and reduce the incidence of respiratory and cardiac arrest. Patients in hospital have other complications including but not limited to stroke, hypotension, septic shock, falls, delirium, pain, nausea, vomiting, depression, mood disturbances, and behavioral issues. Complications in hospitalized patients are frequently associated with delays in identification of clinical deterioration secondary to infrequent or inadequate monitoring. The AVD-M monitor is designed to remotely identify these issues through algorithms that utilize machine vision and artificial intelligence. There are a number of logistical issues in hospitals such as presence of patient in the room, room cleaning, turning of patients in beds, room availability and preparation for the next patient which can be identified through remote, non-contact, video monitoring utilizing machine vision and artificial intelligence. Patients having remote telemedicine visits or remote at home monitoring can have parameters monitored either intermittently, during or after a medical exam, or continuously utilizing their home computer and camera.

Training the AVD-M monitor requires video recordings and patient data from hospitalized patients. De-identification of administratively collected data, data that is collected as part of routine care, would preclude the need for patient consent, and greatly expand the amount of information available for training artificial intelligence systems. Using de-identified patient care data avoids the complication, cost, and time associated with patient consent, making administrative data available for unsupervised learning. De-identification vastly expands the data sets available for unsupervised learning.

The current system, AnonVisioGuard (AVG): Empowering Health Insights Safely. AVG de-identifies facial images allowing unsupervised learning using de-identified PHI data permitting the collection and use of administrative data, collected as part of routine health care, including vital signs and video recordings, for the unsupervised training of machine learning, neural network, deep learning, and other algorithms using de-identified PHI.

Why are datasets that contain facial images necessary? The AVD-M monitor is designed to create a smart hospital or nursing home room that provides continuous or intermittent, remote, non-contact monitoring of vital signs and is able to detect issues with hypoxia, hypoventilation, hypotension, shock, stroke, falls, pain, depression, mood, behavioral issues, delirium, vomiting, obstructive breathing, etc. to reduce the incidence of respiratory and cardiac arrest and other clinical deterioration that can lead to significant morbidity and mortality. The AVD-M monitor can be used in hospitals, nursing homes, home, psychiatric facilities, prisons, airports, waiting rooms, and during telemedicine visits. The AVD-M monitoring can also provide logistical information for hospital management including room occupancy, room readiness for the next patient, room cleaning, ambient noise, light, disturbances that disrupt patient sleep, bed turning, fall prevention etc. Training of the AVD-M monitor requires facial images, images of the patient, vital sign, and other data all of which are PHI. De-identification of the video recordings, and vital sign data allows the use of administrative data, obtained without patient consent, for training of algorithms. It also allows the collection and storage of routine collected data for identification and development of algorithms for rare events such as stroke, delirium, shock, etc.

The Health Insurance Portability and Accountability Act is a 1996 Federal law that restricts access to individuals' private medical information. The HIPAA Privacy Rule was issued by the U.S. Department of Health and Human Services (HHS) to implement the requirement of HIPAA. The Privacy Rule establishes a set of national standards for the protection of certain health information, including who is covered, what information is protected, and how PHI can be used and disclosed. The Privacy Rule also addresses individuals' privacy rights to understand and control how their health information is used. The Office for Civil Rights (OCR) within HHS has responsibility for implementing and enforcing the Privacy Rule with respect to voluntary compliance activities and civil money penalties. Facial images are defined as PHI and are protected by HIPAA.

HIPAA requires that patient consent be obtained for use of PHI, if the patient can be identified. HIPAA allows the use of de-identified patient information for research without patient consent.

How does one collect video files, which are PHI, without patient consent? The simple approach is to de-identify the facial images making them no longer PHI, and making them available for use in research. Artificial intelligence algorithms require large data sets for training. Using de-identified patient information, collected as part of routine healthcare provides this data source but it must be de-identified to allow its use without patient consent.

### Definitions

AVG Anon VisioGuard: A system to de-identify facial images to allow their use in research, development, and training of machine vision systems. AVG anonymizes visual images of patients.

Unsupervised learning: Unsupervised learning, also known as unsupervised machine learning, uses machine learning algorithms to analyze and cluster unlabeled datasets. These algorithms discover hidden patterns or data groupings without the need for human intervention.

Supervised learning refers to a class of systems and algorithms that determine a predictive model using data points with known outcomes. The model is learned by training through an appropriate learning algorithm (such as linear regression, random forests, or neural networks) that typically works through some optimization routine to minimize a loss or error function. Supervised learning is the process of teaching a model by feeding it input data as well as correct output data. This input/output pair is usually referred to as “labeled data.”

Labeled Data: data that has been tagged with one or more labels to provide context or information. Labels can identify properties, characteristics, classifications, or contained objects of the data. Labeled data is useful for certain types of machine learning, such as supervised machine learning, where the model learns from the labels. Data labeling is the process of adding labels to raw data, such as images, text files, or videos.

Administrative Data: data collected as part of routine healthcare and may include many data types.

De-identified health data is patient information that has had personally identifiable information (PII) and PHI removed. Direct identifiers, such as a patient's name, address, medical record information, etc., are removed from the data. The process of de-identification mitigates privacy risks to individuals and allows organizations to share the data without the potential of violating HIPAA. De-identified data can be used in areas such as research, policy assessment, and comparative effectiveness studies. Multiple methods have been used to de-identify facial images. Most methods of facial de-identification degrade the image reducing the accuracy of models based on de-identified facial images. There are reversible methods of de-identification and recovery of the original images. De-identification of medical record data, including radiology images, for use in research is a standard approach.

HIPAA stands for the Health Insurance Portability and Accountability Act. It is a federal law that was enacted in 1996 to establish national standards for the protection of certain health information. The HIPAA Privacy Rule was issued by the U.S. Department of Health and Human Services (HHS) to implement the requirement of HIPAA. The Privacy Rule establishes a set of national standards for the protection of certain health information, including who is covered, what information is protected, and how PHI can be used and disclosed. The Privacy Rule also addresses individuals' privacy rights to understand and control how their health information is used. The Office for Civil Rights (OCR) within HHS has responsibility for implementing and enforcing the Privacy Rule with respect to voluntary compliance activities and civil money penalties.

AVD-M—Audiovisual detection monitor.

Heart Rate—(HR) The rate of the beating of the heart in beats per minute.

Respiratory Rate—(RR) The rate of breathing in breaths per minute.

Tidal Volume—(TV) The volume of air exchanged on a single breath in milliliters per breath.

Pulse Oximetry (SpO₂) The oxygen saturation of arterial blood in percentage.

Systolic Blood Pressure (SBP) The systolic arterial blood pressure in mmHg.

Diastolic Blood Pressure (DBP) The diastolic blood pressure in mmHg.

Mean Arterial Blood Pressure (MAP) The mean blood pressure in mmHg.

Pain—Measured by VAS (Visual Analogue Scale), a dimensionless, subjective measure of pain. Where zero (0) is defined as no pain and ten (10) is defined as the worst pain imaginable. Can also be measured by Critical-Care Pain Observation Tool (CPOT), the possible total score ranges from 0 (no pain) to 8 (maximum pain). The CPOT cutoff score was >2 during nociceptive procedures. A limitation of the CPOT is the lack of sufficient research in delirious critically ill patients. The CPOT includes evaluation of four different behaviors (facial expressions, body movements, muscle tension, and compliance with the ventilator for mechanically ventilated patients or vocalization for nonintubated patients) rated on a scale of zero to two with a total score ranging from 0 to 8. There are multiple other clinical measures of pain that can be used to calibrate the AVD-M pain detection system.

Delirium—Delirium is a serious change in mental abilities. It results in confused thinking and a lack of awareness of someone's surroundings. The disorder usually comes on quickly within hours or a few days. Delirium can often be traced to one or more factors. There are multiple diagnostic algorithms based on four cardinal features of delirium: (1) acute onset and fluctuating course; (2) inattention; (3) disorganized thinking; and (4) altered level of consciousness. The CAM diagnostic algorithm evaluates four key features of delirium: 1) Acute Change in Mental Status with Fluctuating Course, 2) Inattention, 3) Disorganized Thinking, and 4) Altered Level of Consciousness. There are multiple scores for delirium. The Cognitive Test of Delirium (CTD) evaluates five items, each of which receives a score of 0, 2, 4, or 6 points. Thus, the scale has a total of 30 points possible. There is no subdivision described in the literature that correlates the severity levels with the respective CTD values. The Delirium Rating Scale (DRS) 3 is a widely used delirium rating instrument that specifically, sensitively, and reliably measures delirium symptoms as rated by a psychiatrist or trained clinician. The CAM-ICU score is a validated and commonly used score to help monitor patients for the development or resolution of delirium. It is an adaptation of the Confusion Assessment Method (CAM) score for use in ICU patients.

Mood—The conception of mood in cognitive psychology is derived from the analysis of emotion. Mood is considered as a group of persisting feelings associated with evaluative and cognitive states which influence all the future evaluations, feelings and actions. Multiple parameters of mood will be presented including but not limited to: anger, disgust, fear, happiness, sadness, surprise, and neutral.

Patient Call System—Two-way video conferencing between patient and nurse, doctor, or family. Can be used to communicate needs, emergencies, and or requests. Also has speech recognition and text to speech capabilities with natural language processing and text messaging to staff and from staff or family to patient.

Out of Bed Alarm—System to track patient location in room and or in bed. If set, will send alarm if patient attempts to leave the bed or chair or designated location.

Stroke—A stroke occurs when something blocks blood supply to part of the brain or when a blood vessel in the brain bursts. In either case, parts of the brain become damaged or die. A stroke can cause lasting brain damage, long-term disability, or even death. The warning signs of stroke include: Numbness or weakness in the face, arm, or leg; Confusion or trouble speaking or understanding speech; Trouble seeing in one or both eyes; Trouble walking, dizziness, or problems with balance; severe headache with no known cause. The AVD-M system will track patient movement and speech. Changes in patient movement or speech will be evaluated as indication of possible strokes.

Regional Block—Regional anesthesia utilizes local anesthetic to anesthetize areas of the body. The types of regional anesthesia include spinal anesthesia (also called subarachnoid block), epidural anesthesia, and nerve blocks. When local anesthesia is used, motor movement of the affected area may be reduced depending on the location of the block and the concentration of the local anesthetic. Local anesthesia affects nerves based on their myelination and the size of the nerves. Sympathetic nerves, which control local vasodilation, are small fiber and blocked by local anesthesia. Vasodilation, caused by sympathetic blockade, can be detected by changes in skin blood flow, which can be visualized by changes in skin pulsatility signal. The dermatomal level of a regional block will be reported by changes in pulsatility of the skin in a dermatome. Dermatomes are areas of skin, each of which is connected to a single spinal nerve. Together, these areas create a surface map of the body.

Obstructive breathing—Obstruction of the inflow of the upper airway have the following signs. Agitation or fidgeting, Bluish color to the skin (cyanosis), Changes in consciousness, Choking, Confusion, Difficulty breathing, gasping for air, leading to panic, Unconsciousness (lack of responsiveness), Wheezing, crowing, whistling, or other unusual breathing noises indicating breathing difficulty. The tongue is the most common cause of upper airway obstruction, a situation seen most often in patients who are comatose or who have suffered cardiopulmonary arrest. Other common causes of upper airway obstruction include edema of the oropharynx and larynx, trauma, foreign body, and infection. Discordant respiration or obstructive breathing can be identified by abnormal motion of the chest. Partial airway obstruction: breathing labored, gasping or noisy can be caused by some air escaping from the mouth. patient coughing or making a ‘crowing’ noise. Motion of the chest will be tracked by machine vision. The sounds of respiration will be tracked by the microphone and microphone array.

Vomiting—Vomiting (also known as emesis and throwing up) is the involuntary, forceful expulsion of the contents of one's stomach through the mouth and sometimes the nose. Vomiting will be identified by tracking motion of the abdomen, chest, neck, and head using machine vision.

Shivering—Shivering (also called shuddering) is a bodily function in response to cold and extreme fear in warm-blooded animals. When the core body temperature drops, the shivering reflex is triggered to maintain homeostasis. Skeletal muscles begin to shake in small movements, creating warmth by expending energy. Shivering can also be a response to fever, as a person may feel cold. During fever, the hypothalamic set point for temperature is raised. The increased set point causes the body temperature to rise (pyrexia), but also makes the patient feel cold until the new set point is reached. Severe chills with violent shivering are called rigors. Rigors occur because the patient's body is shivering in a physiological attempt to increase body temperature to the new set point. Shivering will be identified by machine vision.

Heart failure (HF), also known as congestive heart failure (CHF), is a syndrome, a group of signs and symptoms caused by an impairment of the heart's blood pumping function. Symptoms typically include shortness of breath, excessive fatigue, and leg swelling. The shortness of breath may occur with exertion or while lying down, and may wake people up during the night. Chest pain, including angina, is not usually caused by heart failure, but may occur if the heart failure was caused by a heart attack. The severity of the heart failure is measured by the severity of symptoms during exercise. Other conditions that may have symptoms similar to heart failure include obesity, kidney failure, liver disease, anemia, and thyroid disease. Congestive heart failure will be identified by machine vision and artificial intelligence analysis utilizing multiple parameters.

Heart Rate Variability (HRV) Heart rate variability (HRV) is the physiological phenomenon of variation in the time interval between heartbeats. It is measured by the variation in the beat-to-beat interval. Other terms used include: “cycle length variability”, “R-R variability” (where R is a point corresponding to the peak of the QRS complex of the ECG wave; and RR is the interval between successive Rs), and “heart period variability”. Heart rate variability will be measured by the variation in the interval of peak signal from skin pulsatility using machine vision.

Predictive Algorithms—Composite variables combining multiple parameters such as heart rate, respiratory rate, SpO₂, blood pressure, and temperature, can be combined together to predict the risk of impending clinical events. The combination of parameters will be used to create parameters that will predict the likelihood of clinical events so that clinical care can be provided in advance of severe deterioration interrupting the chain of events that can lead to respiratory and cardiac arrest.

Cough—A cough is a sudden expulsion of air through the large breathing passages which can help clear them of fluids, irritants, foreign particles and microbes. As a protective reflex, coughing can be repetitive with the cough reflex following three phases: an inhalation, a forced exhalation against a closed glottis, and a violent release of air from the lungs following opening of the glottis, usually accompanied by a distinctive sound. Frequent coughing usually indicates the presence of a disease. Coughing will be detected by sound using the microphone and microphone array.

Temperature—Patient temperature is a standard vital sign measured either in degrees Fahrenheit or degrees Celsius. Normal human body-temperature (normothermia, euthermia) is the typical temperature range found in humans. The normal human body temperature range is typically stated as 36.5-37.5° C. (97.7-99.5° F.).

Depression is a mental state of low mood and aversion to activity. It affects more than 280 million people of all ages (about 3.5% of the global population). Classified medically as a mental and behavioral disorder, the experience of depression affects a person's thoughts, behavior, motivation, feelings, and sense of well-being. The core symptom of depression is said to be anhedonia, which refers to loss of interest or a loss of feeling of pleasure in certain activities that usually bring joy to people. Depressed mood is a symptom of some mood disorders such as major depressive disorder and dysthymia; it is a normal temporary reaction to life events, such as the loss of a loved one; and it is also a symptom of some physical diseases and a side effect of some drugs and medical treatments. It may feature sadness, difficulty in thinking and concentration and a significant increase or decrease in appetite and time spent sleeping. People experiencing depression may have feelings of dejection, hopelessness and suicidal thoughts. It can either be short term or long term. Depression can be measured by patient responses to a PHQ-9 survey tool. The 9-question Patient Health Questionnaire (PHQ-9) is a diagnostic tool introduced in 2001 to screen adult patients in a primary care setting for the presence and severity of depression. It rates depression based on the self-administered Patient Health Questionnaire (PHQ). The PHQ-9 takes less than 3 minutes to complete and simply scores each of the 9 DSM-IV criteria for depression based on the mood module from the original PRIME-MD.

Skin Blood Flow—Hemodynamics are the dynamics of blood flow. The circulatory system is controlled by homeostatic mechanisms of autoregulation, just as hydraulic circuits are controlled by control systems. The hemodynamic response continuously monitors and adjusts to conditions in the body and its environment. Hemodynamics explains the physical laws that govern the flow of blood in the blood vessels. Blood flow ensures the transportation of nutrients, hormones, metabolic waste products, oxygen, and carbon dioxide throughout the body to maintain cell-level metabolism, the regulation of the pH, osmotic pressure and temperature of the whole body, and the protection from microbial and mechanical harm. Blood is a non-Newtonian fluid, and is most efficiently studied using rheology rather than hydrodynamics. Because blood vessels are not rigid tubes, classic hydrodynamics and fluids mechanics based on the use of classical viscometers are not capable of explaining haemodynamics. Skin blood flow will be measured by pulsatile changes in the light reflection from the skin in visible and or infrared spectrum.

Muscle Blood Flow—Blood flow in the muscle underlying skin will be assessed by measurement of pulsatile changes in the light reflection from the skin and subcutaneous tissue in visible and or infrared spectrum.

Shock—Shock is the state of insufficient blood flow to the tissues of the body as a result of problems with the circulatory system. Initial symptoms of shock may include weakness, fast heart rate, fast breathing, sweating, anxiety, and increased thirst. This may be followed by confusion, unconsciousness, or cardiac arrest, as complications worsen. There are multiple types of shock including: septic shock, cardiogenic shock, hypovolemic shock, anaphylactic shock, and neurogenic shock.

Networking—Signals from the AVD-M monitor will be transmitted to the hospital electronic health care record (EHR), centralized monitoring station, individual clinician computers, remote computers, remote hospital locations using standard networking capabilities including but not limited to JSON, HL7, FHIR, and or proprietary formats. Information will also be obtained from the electronic health record that may or may not be included as input parameters in predictive models or displayed.

Eulerian Video Magnification, takes a video sequence as input, separates the individual colors (R, G, B, I, other), followed by temporal filtering to the frames. The resulting signal is then amplified to reveal hidden information such as pulsatility from blood flow in the skin.

Platform—The AVD-M system will consist of either single or multiple electronic cameras with different light sensitivity. It may consist of red-green-blue (RGB) camera(s), infrared camera(s), stereoscopic camera(s), or depth camera(s). It may consist of multiple or singular cameras or a camera that is part of a personal computer, personal tablet computer, or additional camera (web camera). It will also consist of a computer or computers with or without a graphical processor unit (GPU) for array processing. It may or may not have light sources in visible or infrared light. It may have cameras with different light spectrum from standard RGB cameras. It may have wide angle, normal, or telephoto lens with or without autofocusing. It may or may not have a microphone and or microphone array. It may or may not have speakers to generate sound. It may or may not have a display, keyboard, mouse, and networking capabilities. It will have software that is either resident on the computer or hosted on others computers or on the cloud. Analysis of images may be local on the computer, on another local computer, or hosted on the cloud or other servers. The AVD-M system may or may not be able to run software from other companies that provide additional parameters as a platform for monitoring.

Purpose: It is therefore an object of the present system to provide a method of measurement of the above parameters.

Vital signs (also known as “vitals”) are a group of the four to six most crucial medical signs that indicate the status of the body's vital (life-sustaining) functions. These measurements are taken to help assess the general physical health of a person, give clues to possible diseases, and show progress toward recovery. The normal ranges for a person's vital signs vary with age, weight, gender, and overall health. There are four primary vital signs: body temperature, blood pressure, pulse (heart rate), and breathing rate (respiratory rate), often notated as T, BP, HR, and RR. However, depending on the clinical setting, the vital signs may include other measurements called the “fifth vital sign” or “sixth vital sign”.

Vital signs are recorded using the LOINC (Logical Observation Identifiers, Names, and Codes) system. LOINC is a standard, universal coding system that provides a common language for identifying and accepted standard coding system, and is used, among other ways, for exchanging clinical and laboratory observations, measurements, and documents. LOINC is used by health care organizations to code laboratory test orders and results, as well as other clinical measures like vital signs and anthropometric measures. For example, the code 2345-7 is used to identify the amount of glucose measured in a blood test.

Early warning scores have been proposed that combine the individual values of vital signs into a single score. This was done in recognition that deteriorating vital signs often precede cardiac arrest and/or admission to the intensive care unit. Used appropriately, a rapid response team can assess and treat a deteriorating patient and prevent adverse outcomes.

### Algorithms

Unsupervised algorithms: Data that is recorded as part of routine monitoring including video recordings, vital sign recording, hospital record data is de-identified. Images are extracted from the de-identified facial images. Some algorithms use deep learning from these images. Some algorithms extract predefined features from the images and use neural network or other algorithms. Data indicating the parameter being identified is associated with images using neural network, deep learning networks, or other learning algorithms. Unsupervised learning can utilize parameters such as heart rate, respiratory rate, pulse oximetry, blood pressure, etc, collected by standard routine and specialized invasive monitors as the outcome variable for learning algorithms.

Approach to measurement: The generalized approach to measurement is to have a video signal from RGB or IR or Depth cameras or stereoscopic cameras or cameras of other frequencies. Different algorithms require different approaches to measurement. The camera system collects an image. Algorithms on the computer identify the patient separate from the background image. The patient's face is identified using facial recognition algorithms such as but not limited to Haar Cascade based algorithms. The patient may or may not then be identified by facial recognition to ensure the correct person is monitored. Pulsatility in the skin is then assessed by Eulerian magnification algorithms or other techniques in one or multiple colors such as red, green, blue, and or infrared. A pulsatile signal from in the skin may assessed in one or multiple colors (R, G, B, I, other). One embodiment utilizes the area above the eyebrows on the face for measurement of heart rate, SpO₂, and Respiratory rate. Signals from the chest or other areas may also be used for respiratory rate. Skin pulsatility imaging or motion of the chest utilizing depth imagine or stereoscopic imaging can be used to measure respiratory rate, tidal volume, and breathing patterns. Imaging can be achieved in the dark with infrared. If additional light is needed, lights of appropriate color and intensity can be turned on including but not limited to red, green, blue, infrared. Lights may or may not be targeted or diffuse. Lighting may be controlled automatically by the system when needed or on control of clinical staff or patient.

Heart Rate: Can be measured by time domain or frequency domain or both analysis of pulsatile signals from the skin from one or more of the cameras (R, G, B, I, other) in one or more colors. A standard location for measurement of heart rate is an area above the eyebrows on the forehead but other sites can be used to provide signal.

Respiratory Rate: Can be measured by time domain or frequency domain or both analysis of pulsatile signals from the skin from one or more of the cameras (R, G, B, I, other) in one or more colors. A standard location for measurement of respiratory rate is an area above the eyebrows on the forehead or the chest. Other areas can be used to provide the signal. Depth imaging of the chest can be used with or without or in combination with Eulerian magnification to provide input to the respiratory rate algorithm.

Tidal Volume: Dimensions of the chest can be obtained from the video image from RGB, IR, Depth, or stereoscopic cameras. Motion of the chest wall can be obtained from the depth or stereoscopic cameras. An algorithm that either explicitly calculates tidal volume from chest dimensions and chest wall motion can be used or an empiric model can be developed using statistical modelling, neural network, or deep learning algorithms. The model would be trained to produce tidal volume from single or multiple video inputs.

SpO₂: Video signals from the RGB and IR camera(s) would be decomposed into individual colors. The peak and trough of each color channel would be identified. The ratios of (RedPeak/RedTrough)/(IRPeak/IRTrough), (GreenPeak/GreenTrough)/(IRPeak/IRTrough), (BluePeak/BlueTrough)/(IRPeak/IRTrough), (RedPeak/RedTrough)/(GreenPeak/GreenTrough), (RedPeak/RedTrough)/(BluePeak/BlueTrough), (GreenPeak/GreenTrough)/(BluePeak/BlueTrough), would be calculated. A calibration parameter would be adjusted for each individual color ratio. One or multiple ratios would be utilized based on signal quality, calibration, and lighting conditions.

Perfusion: The pulsatile color or IR signal will be calculated from the surface of the skin and mapped into a pulsatility image. The IR color signal can produce the image in the dark or through clothing or blankets. The level and gain of the displayed signal can be adjusted manually or automatically to produce the best pulsatility image. Areas of interest can be identified and highlighted such as a plastic surgery free flap or a limb with possible ischemia or compartment syndrome.

Systolic Blood Pressure: The pulsatile color or IR signal will be calculated from the surface of the skin. Signal averaging will be performed based on the peak or trough signal. A signal averaged pulsatile waveform will be produced. An explicit algorithm based on wave reflection or other approach can be produced to map the pulsatile signal to systolic blood pressure. In an alternative embodiment, a neural network model can be used to create a predictive model from pulsatile signal to systolic blood pressure. In an alternative embodiment, a deep learning model will be produced mapping the video image in RGB or IR to systolic blood pressure with or without extracting parameters or doing prefiltering or signal averaging.

Diastolic Blood Pressure: The pulsatile color or IR signal will be calculated from the surface of the skin. Signal averaging will be performed based on the peak or trough signal. A signal averaged pulsatile waveform will be produced. An explicit algorithm based on wave reflection or other approach can be produced to map the pulsatile signal to diastolic blood pressure. In an alternative embodiment, a neural network model can be used to create a predictive model from pulsatile signal to diastolic blood pressure. In an alternative embodiment, a deep learning model will be produced mapping the video image in RGB or IR to diastolic blood pressure with or without extracting parameters or doing prefiltering or signal averaging.

Mean Arterial Blood Pressure: The pulsatile color or IR signal will be calculated from the surface of the skin. Signal averaging will be performed based on the peak or trough signal. A signal averaged pulsatile waveform will be produced. An explicit algorithm based on wave reflection or other approach can be produced to map the pulsatile signal to mean arterial blood pressure. In an alternative embodiment, a neural network model can be used to create a predictive model from pulsatile signal to mean arterial blood pressure. In an alternative embodiment, a deep learning model will be produced mapping the video image in RGB or IR to systolic blood pressure with or without extracting parameters or doing prefiltering or signal averaging.

Pain: Video signals in RGB, IR, or other colors will be recorded. A neural network or AI algorithm will identify the patient's face. Multiple locations will be identified on the patients face and facial action units identified. Facial action unit changes will then be mapped by neural network or other AI algorithm to pain measured either by VAS (Visual Analogue Scale) or Critical-Care Pain Observation Tool (CPOT), or both, or another pain measurement tool. In an alternative embodiment, the image of the patient's face in RGB, IR, or other color spectrum will be fed into a deep learning algorithm and matched to a pain score. In an alternative embodiment, images of the face, and or body, limbs, and or sounds, and or vital signs (HR, RR, etc) will be fed into a deep learning or other statistical model or neural network trained by a pain measurement tool. The combination of vital signs and facial motions from action units and other signals may be analyzed by individual or multiple neural networks or other analytic tools to predict a pain score.

Pain measurement tools that may be used include but are not limited to: Alder Hey Triage Pain Score, Behavioral Pain Scale (BPS), Brief Pain Inventory (BPI), Checklist of Nonverbal Pain Indicators (CNPI), Clinical Global Impression (CGI), COMFORT scale, Color Scale for Pain, Critical-Care Pain Observation Tool (CPOT), Dallas Pain Questionnaire, Descriptor differential scale (DDS), Dolorimeter Pain Index (DPI), Edmonton Symptom Assessment System, Face Legs Activity Cry Consolability scale, Faces Pain Scale-Revised (FPS-R), Global Pain Scale, Lequesne algofunctional index: a composite measure of pain and disability, with separate self-report questionnaires for hip and knee OA (osteoarthritis), Mankoski Pain Scale, McGill Pain Questionnaire (MPQ), Multiple Pain Rating Scales, Neck Pain and Disability Scale-NPAD, Numerical 11 point box (BS-11), Numeric Rating Scale (NRS-11), Oswestry Disability Index, Palliative Care Outcome Scale (PCOS), Roland-Morris Back Pain Questionnaire, Support Team Assessment Schedule (STAS), Wharton Pain and Impairment Numeric Scale (Wharton PAIN Scale), Wong-Baker FACES Pain Rating Scale, Visual analog scale (VAS), Abbey pain scale for people with end-stage dementia, AUSCAN: Disease-Specific, to assess hand osteoarthritis outcomes., Colorado

Behavioral Numerical Pain Scale (for sedated patients), CPOT For those who can't self report, Osteoarthritis Research Society International-Outcome Measures in Rheumatoid Arthritis Clinical Trials (OARSI-OMERACT) Initiative, New OA Pain Measure: Disease-Specific, Osteoarthritis Pain, Oucher Scale for Pediatrics, Pain Assessment in Advanced Dementia (PAINAD), Pediatric Pain Questionnaire (PPQ) for measuring pain in children, Premature Infant Pain Profile (PIPP) for measuring pain in premature infants, Schmidt Sting Pain Index and Starr sting pain scale both for insect stings, WOMAC: Disease-Specific, to assess knee osteoarthritis outcomes.

Delirium: A neural network, statistical model, or other algorithm will take video signals, and or sounds and map those images to a measurement of delirium. Patient limb motions, verbalizations, position in the bed, limb motions, eye movement, and other inputs may be used singularly or in combination to be analyzed by a neural network, deep learning network, statistical model, or other analytic tool to predict a delirium score. Additional data may or may not be added to the predictive model from the electronic health care record including but not limited to medications administered, past medical history, age, pre-existing medical conditions, sedatives administered, etc. Delirium will be match to a diagnosis of delirium and may or may not be measure with an objective tool including but not limited to: Richmond Agitation and Sedation Scale (RASS)—highly sensitive and specific for diagnosing delirium in older patients, Observational Scale of Level of Arousal (OSLA)—highly sensitive and specific for diagnosing delirium in older patients, Confusion Assessment Method (CAM), Delirium Observation Screening Scale (DOS), Nursing Delirium Screening Scale (Nu-DESC), Recognizing Acute Delirium As part of your Routine (RADAR), 4AT (4 A's Test), Delirium Diagnostic Tool-Provisional (DDT-Pro), also for subsyndromal delirium.

Delirium Risk Factor Prediction: Risk factors for delirium will be assessed from the environment including sound levels by time of day. Light levels by time of day. Disturbances including sound, light, alarms, clinical visits, etc during normal sleeping hours. The most important predisposing factors for delirium will be obtained from the medical record including:
65 or more years of age
Male sex
Cognitive impairment/dementia
Physical comorbidity (biventricular failure, cancer, cerebrovascular disease)
Psychiatric comorbidity (e.g., depression)
Sensory impairment (vision, hearing)
Functional dependence (e.g., requiring assistance for self-care or mobility)
Dehydration/malnutrition
Drugs and drug-dependence
Alcohol dependence

### Precipitating Factors:

Prolonged sleep deprivation.
Environmental, physical/psychological stress
   Inadequately controlled pain
   Admission to an intensive care unit
   Immobilization, use of physical restraints [25]
   Urinary retention, use of bladder catheter,
   Emotional stress
   Severe constipation/fecal impaction

### Medications: [26] [27]

Sedatives
(benzodiazepines, opioids), anticholinergics, dopaminergics, corticosteroids, polypharmacy
General anesthetic
Substance intoxication or withdrawal

### Primary Neurologic Diseases:

Severe drop in blood pressure, relative to the patient's normal blood pressure (orthostatic hypotension) resulting in inadequate blood flow to the brain (cerebral hypoperfusion)
Stroke/Transient ischemic attack (TIA)
Intracranial bleeding
Meningitis, encephalitis

### Concurrent Illness:

Infections-especially respiratory (e.g. pneumonia, COVID-19 [28]) and urinary tract infections
Iatrogenic complications
Hypoxia, hypercapnea, anemia
Poor nutritional status, dehydration, electrolyte imbalances, hypoglycemia
Shock, heart attacks, heart failure
Metabolic derangements (e.g. SIADH, Addison's disease, hyperthyroidism,)
Chronic/terminal illness (e.g. cancer)
Post-traumatic event (e.g. fall, fracture)
Mercury poisoning (e.g. Erethism)

### Surgery:

Cardiac, orthopedic, prolonged cardiopulmonary bypass, thoracic surgeries

A delirium risk score will be presented. Risk factors that can be reduced by changes in care plan such as reduced disturbances, light levels, sounds, medication choices will be identified.

Mood—Video signals in RGB, IR, or other colors will be recorded. A neural network or AI algorithm will identify the patient's face. Facial identification will be performed to ensure the correct patient is analyzed. Multiple locations will be identified on the patients face and facial action units identified. Facial action unit changes will then be mapped by neural network or other AI algorithm to mood. Multiple parameters of mood will be displayed including but not limited to: anger, disgust, fear, happiness, sadness, surprise, depression, and neutral. Mood will be validated by patient report for the training session. Depression will be validated by PHQ-9 or other depression assessment tools.

Out of Bed Alarm: Video images of the patient's room will be obtained in RGB, IR, depth, or stereoscopic, or other color. Machine vision will be used to identify the patient's bed or other location. In an alternative embodiment, the area the patient is supposed to be located in can be identified by location on a computer screen with delimiters. Delimiters can be either automatically placed by the machine vision system or manually by clinical or other staff. The patient will then be identified in the bed or other area. If the patient's arms, legs, torso, or other identified area passes or begins to pass beyond the delimiter a notification will be flagged or sent or an alarm sounded depending on settings. Video imaging may or may not be sent to the central control station by settings.

Stroke: One of the most common types of stroke are strokes that affect the motor cortex. Patient motion of the limbs (arms and legs) and face will be tracked and time. Time for last motion of each limb will be recorded. Time for motion of the face will be recorded. Time for last intelligible speech command will be recorded. A setting in the system will be programmable of time to notify if an area of the body, which previously had motion, is no longer moving. Such as time since left arm last moved. Time since right arm last moved. Clinical staff can set the time for an alert or it can be adjusted automatically to reduce false alarm rates. If a patient has a preexisting neurologic abnormality such as prior stroke, baseline mobility will be recorded and areas with normal motion tracked. If a patient has a preexisting dysarthria or is aphasic, the system will note the pre-existing condition and alarms will be set accordingly. The system will provide multiple factors, last identified movement of the suspected area, such as time left arm moved last. The system will identify the time since motion or function was normal. The system will have a recording of baseline motor and speech activity for reference. The system can be set to alert clinical staff to a change from baseline mobility after a specified time interval.

Regional Block: Local anesthetics effect nerves based on nerve size, myelination, and local anesthetic concentration. Because sympathetic nerves are small diameter fibers, they are rapidly and easily blocked by local anesthetic concentrations. Sympathetic nerve block effects local blood flow can cause vasodilation. Vasodilation effects pulsatility imaging. The pulsatile image of areas of the body will be calculated. Variations in pulsatility imaging of the skin will be used to map the dermatomal level of the sympathetic block caused by the local anesthetic. Dermatomal maps and or levels will be presented for assessment of regional block.

Obstructive Breathing: Dimensions of the chest can be obtained from the video image from RGB, IR, Depth, or stereoscopic cameras. Motion of the chest wall can be obtained from the depth or stereoscopic cameras. An algorithm that either explicitly calculate tidal volume and identifies obstructive breathing from chest dimensions and chest wall motion can be used or an empiric model can be developed using statistical modelling, neural network, or deep learning algorithms. The model would be trained to identify obstructive breathing from single or multiple video inputs. Obstructive breathing can be identified by paradoxical motion of the chest wall and supraclavicular area. Paradoxical motion of the chest and or low tidal volumes will be identified. Dyscoordinated breathing patterns will be identified and warnings of possible residual nondepolarizing neuromuscular blockade will be identified by paradoxical or abnormal breathing and or motor activity patterns.

Vomiting: Dimensions of the chest can be obtained from the video image from RGB, IR, Depth, or stereoscopic cameras. Motion of the chest wall, head, face, etc can be obtained from the depth or stereoscopic cameras. An algorithm that either explicitly identifies vomiting from chest wall and head motion can be used or an empiric model can be developed using statistical modelling, neural network, or deep learning algorithms. The model would be trained to identify vomiting from single or multiple video inputs.

Shivering: Motion of the chest, limbs, muscles can be obtained from the video image from RGB, IR, Depth, or stereoscopic cameras. Eulerian magnification or other techniques can be used to identify rapid motions. Motion of the chest wall, head, face, limbs etc can be obtained from the depth or stereoscopic cameras. An algorithm that either explicitly identifies shivering from patient motion can be used or an empiric model can be developed using statistical modelling, neural network, or deep learning algorithms. The model would be trained to identify shivering from single or multiple video inputs.

Congestive Heart Failure (CHF): Congestive heart failure presents a constellation of symptoms. Congestive heart failure (CHF), is a syndrome, a group of signs and symptoms caused by an impairment of the heart's blood pumping function. Symptoms typically include shortness of breath, excessive fatigue, and leg swelling. The shortness of breath may occur with exertion or while lying down, and may wake people up during the night. The severity of the heart failure is measured by the severity of symptoms during exercise. Other conditions that may have symptoms similar to heart failure include obesity, kidney failure, liver disease, anemia, and thyroid disease. Common causes of heart failure include coronary artery disease, heart attack, high blood pressure, atrial fibrillation, valvular heart disease, excessive alcohol consumption, infection, and cardiomyopathy. These cause heart failure by altering the structure or the function of the heart or in some cases both. There are different types of heart failure: right-sided heart failure, which affects the right heart, left-sided heart failure, which affects the left heart, and biventricular heart failure, which affects both sides of the heart. Left-sided heart failure may be present with a reduced ejection fraction or with a preserved ejection fraction. Heart failure is not the same as cardiac arrest, in which blood flow stops completely due to the failure of the heart to pump effectively. Diagnosis is based on symptoms, physical findings, and echocardiography. Blood tests, and a chest x-ray may be useful to determine the underlying cause. CHF can be identified by images from the video system extracting a pattern of parameters including heart rate, respiratory rate, blood pressure, and perfusion. A multi-parameter model will be produced using neural networks, or statistical modeling, or deep learning from images and other parameters such as vital signs measured by AVD-M and or from the electronic health record. The CHF model will be trained using AI, neural networks, deep learning, statistical modeling, or other analytic techniques.

Heart Rate Variability (HRV): Can be measured by time domain or frequency domain or both. Analysis of pulsatile signals from the skin from one or more of the cameras (R, G, B, I, other) in one or more colors will be used. A standard location for measurement of heart rate variability is an area above the eyebrows on the forehead but other sites can be used to provide signal. The variation in the time between peaks or between troughs can be used to measure HRV. HRV is a predictor of sympathetic tone and may be used in other algorithms to predict outcomes.

Predictive Algorithms: There is significant morbidity and mortality from a respiratory and or cardiac arrest. Review of vital sign data prior to a cardiac arrest in hospital frequently identifies a slow and prolonged deterioration leading to the cardiac arrest. Some clinical events such as arrythmias, pulmonary emboli, or embolic stroke are sudden events that are difficult to predict. But many clinical events have a prolonged temporal pattern where if the problem is identified early, the progression of events leading to a possible respiratory or cardiac arrest can be stopped avoiding significant morbidity and mortality. To identify this progression of events leading to respiratory and cardiac arrest continuous or frequent monitoring of vital signs combined with analysis of the combination of the vital sign data is needed. The AVD-M system provides continuous measurement of multiple vital signs (HR, RR, SpO₂, BP, etc). Statistical, neural network, deep learning, AI, or other multiparameter models based on data from the AVD-M system, and or electronic health care record (EHR) data will be used to predict the probability of respiratory or cardiac arrest, code blue, and or rapid response calls. A multidimensional model will provide a dimensionless parameter that predicts impending clinical events. Data from EHR records and or AVD-M data will be used to train the predictive model.

Psychiatric Monitoring: Patients in in-patient psychiatric wards need to be monitored on a continuous and or frequent basis. Remote, non-contact monitoring, where the lack of leads reduces the risk of suicide or suicide attempts would reduce risk. Infrared monitoring that does not disturb sleep will reduce risk, disturbances, and improve staff and patient safety. HR, RR, BP, temperature, mood, depression, activity will be monitored continuously with no sensors on the patients.

Cough: Dimensions of the chest can be obtained from the video image from RGB, IR, Depth, or stereoscopic cameras. Motion of the chest wall, head, face, etc can be obtained from the depth or stereoscopic cameras. An algorithm that either explicitly identifies and identifies cough from chest wall and head motion can be used or an empiric model can be developed using statistical modelling, neural network, or deep learning algorithms. The model would be trained to identify cough from single or multiple video inputs.

Temperature: Patient temperature can be measured from infrared imaging of the body. Distribution of patient skin temperature can be presented visually to show peripheral vasoconstriction or vasodilation as part of perfusion imaging, vasodilatory shock detection, and other clinical presentations.

Depression: Video signals in RGB, IR, or other colors will be recorded. A neural network or AI algorithm will identify the patient's face. Facial identification will be performed to ensure the correct patient is analyzed. Multiple locations will be identified on the patients face and facial action units identified. Facial action unit changes will then be mapped by neural network or other AI algorithm or other statistical model to mood. Multiple parameters of mood will be displayed including but not limited to: anger, disgust, fear, happiness, sadness, surprise, depression, and neutral. Mood will be validated by patient report for the training session. Psychomotor retardation, speech patterns, lack of speech, speech content, and body movements may be added to multiparameter models of depression. Depression will be validated by PHQ-9 or other depression assessment tools.

Skin blood flow: The pulsatile color or IR signal will be calculated from the surface of the skin and mapped into a pulsatility image. The IR color signal can produce the image in the dark or through clothing or blankets. The level and gain of the displayed signal can be adjusted manually or automatically to produce the best pulsatility image providing skin blood flow. Areas of interest can be identified and highlighted such as a plastic surgery free flap or a limb with possible ischemia or compartment syndrome.

Muscle blood flow: Muscle blood flow will be inferred from signals from the skin and deeper structures as skin blood flow reflects underlying muscle blood flow. The pulsatile color or IR signal will be calculated from the surface of the skin and mapped into a pulsatility image. The IR color signal can produce the image in the dark or through clothing or blankets. The level and gain of the displayed signal can be adjusted manually or automatically to produce the best pulsatility image providing skin and muscle blood flow. Areas of interest can be identified and highlighted such as a plastic surgery free flap or a limb with possible ischemia or compartment syndrome.

Shock: There are multiple types of shock including vasodilatory shock, cardiogenic shock, hypovolemic shock, anaphylactic shock, neurogenic shock, septic shock. Each type of shock has a variation in the clinical presentation. For example, vasodilatory shock has increased skin perfusion. Cardiogenic shock will have vasoconstriction with poor peripheral perfusion. Hypovolemic shock will have low blood pressure and vasoconstriction and a likely source of bleeding or volume loss. Anaphylactic shock may have cutaneous and or respiratory symptoms. Neurogenic shock will likely be vasodilatory and may have minimal muscle activity or motion. Septic shock may have the production of nitric oxide which increases the methemoglobin levels from the routine less than 0.3% to greater than 1.5%. Increases in methemoglobin levels will change skin tone towards grey or darker color in constellation with hypotension, poor perfusion, tachycardia, and increases in temperature. Methemoglobin (MetHb) is a dysfunctional form of hemoglobin that is incapable of transporting oxygen, thus reducing blood oxygenation and potentially inducing tissue hypoxemia. Because, AVD-M monitoring has four colors (R, G, B, I) it can be calibrated to detect methemoglobin levels in patients. Combinations of parameters which may include or not include: HR, RR, BP, temperature, perfusion, temperature mapping, perfusion mapping, SpO₂, hemoglobin levels will be used to detect and diagnose shock and different types of shock: vasodilatory shock, cardiogenic shock, hypovolemic shock, anaphylactic shock, neurogenic shock, septic shock. Statistical modeling, neural networks, deep learning, or empiric models, with or without additional information from the electronic health record will be used to identify and or diagnose shock and or type of shock.

Smart Alarms: The sensitivity and specificity of data from a monitoring system must be sufficient and the frequency of false alarms low enough that the information is clinically useful to staff. Smart alarm technology will be used that combines multiple parameters, checking of data, improvements in signal to noise, and artificial intelligence to reduce the incidence and frequency of false alarms while maintaining acceptable sensitivity and specificity.

Platform: The AVD-M system has camera(s) including but not limited to RGB, IR, Depth, stereoscopic in addition to microphone(s) or microphone arrays, speaker, monitor, and input and output devices possibly including lights of different color spectrum including but not limited to red, green, blue, white, infrared, etc. This monitoring platform has a computer, software, with or without a graphical processor unit (GPU), and networking capabilities. The hardware and software creates a platform for medical monitoring that can provide input and output capabilities for other software utilizing the same platform. Natural language processing software, predictive models, models that combine data from the AVD-M sensors with data from the electronic health care record (EHR), and software that extracts other parameters or extract parameters with better accuracy, sensitivity, specificity, lower false alarm rates, etc may be hosted on the AVD-M platform. AVD-M software may also be run on patient's home computers utilizing the built in or external camera, microphones, and speakers to provide data for telemedicine visits or home health monitoring.

By extension, this or a similar system might also be used in the hospitals, nursing homes, operating rooms, recovery rooms, intensive care units, step down units, wards, hotels, home, schools, psychiatric facilities, airports, waiting rooms, transportation hubs, buses, planes, trains, or other public or private areas.

It is a further object of the present system to provide a simple method for monitoring people and providing information without physical contact with or sensors on the person. The present system is for a system capable of continuously monitoring people and providing multiple parameters including but not limited to: Heart rate, respiratory rate, oxygen saturation, Blood pressure, tidal volume, perfusion, pain, delirium, mood, patient call and communication, speech recognition, out of bed alarm, stroke, regional block level, obstructive breathing, vomiting, shivering, congestive heart failure, heart rate variability, predictive algorithms, cough, temperature, depression, skin blood flow, muscle blood flow, shock, and a platform for monitoring.

### An Additional Field of System:

AVD-M monitoring can be used in transportation settings such as cars, planes, buses, trains, or other forms of transportation to assess function of the operator as well as presence of persons in the vehicle such as infants to avoid the hot car death scenario.

### Detailed Description of the Preferred Embodiments

In the preferred embodiment, there is a camera array with red, green, blue (RGB) camera, infrared (IR) camera, depth camera. There is a microphone array and speaker. There is a display, In an alternative embodiment there are fewer cameras or cameras of different types such as cameras sensitive to other spectrum of light or stereoscopic cameras or a lidar array. In another alternative embodiment AVD-M monitoring is achieved with the camera on a patient's home computer and the software is run on the home computer or on a cloud or remote computer. The software only model may use whatever camera is available at the distant location such as RGB, IR, Depth, or separate web camera, or camera arrays.

Among other things, the AVD-M monitor provides a platform for monitoring utilizing a camera or cameras or camera array consisting of but not limited to a red, green, blue, infrared, depth, and or stereoscopic, or camera in other frequency ranges, and a microphone or microphone array, a computer, other input output devices, and or a graphical processor unit, and software either resident on the computer or remotely analyzing the input output data from the computer. The sensors detect the patient, identify the patient, locate areas of the body to be monitored, and calculate output parameters continuously or intermittently, and provide clinical information, alarms, and vital sign status of patients. The AVD-M system has networking capabilities to obtain and transmit information to the electronic health care record (EHR), and clinicians, families, and hospital administration as required.

A means for remote, non-contact monitoring of patients utilizing camera(s) and or camera arrays, microphones and or microphone arrays, speakers, monitors, networking, computer, and software to provide vital sign and other information for clinical care.

An apparatus for remote, non-contact monitoring of patients utilizing camera(s) and or camera arrays, microphones and or microphone arrays, speakers, monitors, networking, computer, and software to provide vital sign, logistical, and other information for clinical care.

The following references are incorporated in their entirety:
1. van Galen, L. S., et al., Delayed Recognition of Deterioration of Patients in General Wards Is Mostly Caused by Human Related Monitoring Failures: A Root Cause Analysis of Unplanned ICU Admissions. PLOS One, 2016. 11 (8): p. e0161393.
2. Jeong Y, Y. S., Kim Y, Shim W, De-Identification of Facial Features in Magnetic Resonance Images: Software Development Using Deep Learning Technology. J Med Internet Res, 2020. 22.
3. Roguljić, M., et al., What Patients, Students and Doctors Think About Permission to Publish Patient Photographs in Academic Journals: A Cross-Sectional Survey in Croatia. Sci Eng Ethics, 2020. 26 (3): p. 1229-1247.
4. Kushida, C. A., et al., Strategies for de-identification and anonymization of electronic health record data for use in multicenter research studies. Med Care, 2012. 50 Suppl (Suppl): p. S82-101.
5. Mazura, J. C., et al., Facial recognition software success rates for the identification of 3D surface reconstructed facial images: implications for patient privacy and security. J Digit Imaging, 2012. 25 (3): p. 347-51.
6. Bastian, L., et al., DisguisOR: holistic face anonymization for the operating room. Int J Comput Assist Radiol Surg, 2023. 18 (7): p. 1209-1215.
7. Sahlsten, J., et al., Segmentation stability of human head and neck cancer medical images for radiotherapy applications under de-identification conditions: Benchmarking data sharing and artificial intelligence use-cases. Front Oncol, 2023. 13: p. 1120392.
8. Kim, J. and N. Park, De-Identification Mechanism of User Data in Video Systems According to Risk Level for Preventing Leakage of Personal Healthcare Information. Sensors (Basel), 2022. 22 (7).
9. Dong, B., et al., Private Face Image Generation Method Based on De-identification in Low Light. Comput Intell Neurosci, 2022. 2022: p. 5818180.
10. Preston, F. G., et al., Informed Consent In Facial Photograph Publishing: A Cross-sectional Pilot Study To Determine The Effectiveness Of De-identification Methods. J Empir Res Hum Res Ethics, 2022. 17 (3): p. 373-381.
11. Lin, J., Y. Li, and G. Yang, FPGAN: Face de-identification method with generative adversarial networks for social robots. Neural Netw, 2021. 133: p. 132-147.
12. Gong, M., et al., Disentangled Representation Learning for Multiple Attributes Preserving Face De-identification. IEEE Trans Neural Netw Learn Syst, 2022. 33 (1): p. 244-256.
13. Engelstad, M. E., et al., De-identification of facial images using composites. J Oral Maxillofac Surg, 2011. 69 (12): p. 3026-31.
14. Gross, R., et al., Model-Based de-identification of facial images. AMIA Annu Symp Proc, 2008. 2008: p. 262.
15. Pan, Y. L., J. C. Chen, and J. L. Wu, Towards a Controllable and Reversible Privacy Protection System for Facial Images through Enhanced Multi-Factor Modifier Networks. Entropy (Basel), 2023. 25 (2).
16. Bischoff-Grethe, A., et al., A technique for the de-identification of structural brain MR images. Hum Brain Mapp, 2007. 28 (9): p. 892-903.
17. Leung, K. Y., et al., IT Infrastructure to support the secondary use of routinely acquired clinical imaging data for research. Neuroinformatics, 2015. 13 (1): p. 65-81.

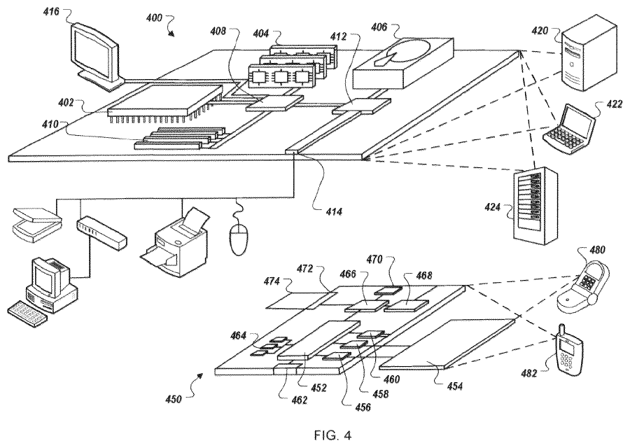

FIG. 4 shows an example of a computing device 400 and an example of a mobile computing device that can be used to implement the techniques described here. The computing device 400 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the systems described and/or claimed in this document.

In FIG. 4, the computing device 400 includes a processor 402, a memory 404, a storage device 406, a high-speed interface 408 connecting to the memory 404 and multiple high-speed expansion ports 410, and a low-speed interface 412 connecting to a low-speed expansion port 414 and the storage device 406. Each of the processor 402, the memory 404, the storage device 406, the high-speed interface 408, the high-speed expansion ports 410, and the low-speed interface 412, are interconnected using various busses, and can be mounted on a common motherboard or in other manners as appropriate. The processor 402 can process instructions for execution within the computing device 400, including instructions stored in the memory 404 or on the storage device 406 to display graphical information for a GUI on an external input/output device, such as a display 416 coupled to the high-speed interface 408. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 404 stores information within the computing device 400. In some implementations, the memory 404 is a volatile memory unit or units. In some implementations, the memory 404 is a non-volatile memory unit or units. The memory 404 can also be another form of computer-readable medium, such as a magnetic or optical disk.

The storage device 406 is capable of providing mass storage for the computing device 400. In some implementations, the storage device 406 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 404, the storage device 406, or memory on the processor 402.

The high-speed interface 408 manages bandwidth-intensive operations for the computing device 400, while the low-speed interface 412 manages lower bandwidth-intensive operations. Such allocation of functions is exemplary only. In some implementations, the high-speed interface 408 is coupled to the memory 404, the display 416 (e.g., through a graphics processor or accelerator), and to the high-speed expansion ports 410, which can accept various expansion cards (not shown). In the implementation, the low-speed interface 412 is coupled to the storage device 406 and the low-speed expansion port 414. The low-speed expansion port 414, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 400 can be implemented in a number of different forms, as shown in the figure. For example, it can be implemented as a standard server 420, or multiple times in a group of such servers. In addition, it can be implemented in a personal computer such as a laptop computer 422. It can also be implemented as part of a rack server system 424. Alternatively, components from the computing device 400 can be combined with other components in a mobile device (not shown), such as a mobile computing device 450. Each of such devices can contain one or more of the computing device 400 and the mobile computing device 450, and an entire system can be made up of multiple computing devices communicating with each other.

The mobile computing device 450 includes a processor 452, a memory 464, an input/output device such as a display 454, a communication interface 466, and a transceiver 468, among other components. The mobile computing device 450 can also be provided with a storage device, such as a micro-drive or other device, to provide additional storage. Each of the processor 452, the memory 464, the display 454, the communication interface 466, and the transceiver 468, are interconnected using various buses, and several of the components can be mounted on a common motherboard or in other manners as appropriate.

The processor 452 can execute instructions within the mobile computing device 450, including instructions stored in the memory 464. The processor 452 can be implemented as a chipset of chips that include separate and multiple analog and digital processors. The processor 452 can provide, for example, for coordination of the other components of the mobile computing device 450, such as control of user interfaces, applications run by the mobile computing device 450, and wireless communication by the mobile computing device 450.

The processor 452 can communicate with a user through a control interface 458 and a display interface 456 coupled to the display 454. The display 454 can be, for example, a TFT (Thin-Film-Transistor Liquid Crystal Display) display or an OLED (Organic Light Emitting Diode) display, or other appropriate display technology. The display interface 456 can comprise appropriate circuitry for driving the display 454 to present graphical and other information to a user. The control interface 458 can receive commands from a user and convert them for submission to the processor 452. In addition, an external interface 462 can provide communication with the processor 452, so as to enable near area communication of the mobile computing device 450 with other devices. The external interface 462 can provide, for example, for wired communication in some implementations, or for wireless communication in other implementations, and multiple interfaces can also be used.

The memory 464 stores information within the mobile computing device 450. The memory 464 can be implemented as one or more of a computer-readable medium or media, a volatile memory unit or units, or a non-volatile memory unit or units. An expansion memory 474 can also be provided and connected to the mobile computing device 450 through an expansion interface 472, which can include, for example, a SIMM (Single In Line Memory Module) card interface. The expansion memory 474 can provide extra storage space for the mobile computing device 450, or can also store applications or other information for the mobile computing device 450. Specifically, the expansion memory 474 can include instructions to carry out or supplement the processes described above, and can include secure information also. Thus, for example, the expansion memory 474 can be provide as a security module for the mobile computing device 450, and can be programmed with instructions that permit secure use of the mobile computing device 450. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 464, the expansion memory 474, or memory on the processor 452. In some implementations, the computer program product can be received in a propagated signal, for example, over the transceiver 468 or the external interface 462.

The mobile computing device 450 can communicate wirelessly through the communication interface 466, which can include digital signal processing circuitry where necessary. The communication interface 466 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 468 using a radio-frequency. In addition, short-range communication can occur, such as using a Bluetooth, WiFi, or other such transceiver (not shown). In addition, a GPS (Global Positioning System) receiver module 470 can provide additional navigation- and location-related wireless data to the mobile computing device 450, which can be used as appropriate by applications running on the mobile computing device 450.

The mobile computing device 450 can also communicate audibly using an audio codec 460, which can receive spoken information from a user and convert it to usable digital information. The audio codec 460 can likewise generate audible sound for a user, such as through a speaker, e.g., in a handset of the mobile computing device 450. Such sound can include sound from voice telephone calls, can include recorded sound (e.g., voice messages, music files, etc.) and can also include sound generated by applications operating on the mobile computing device 450.

The mobile computing device 450 can be implemented in a number of different forms, as shown in the figure. For example, it can be implemented as a cellular telephone 480. It can also be implemented as part of a smart-phone 482, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of the disclosed technology or of what may be claimed, but rather as descriptions of features that may be specific to particular embodiments of particular disclosed technologies. Certain features that are described in this specification in the context of separate embodiments can also be implemented in combination in a single embodiment in part or in whole. Conversely, various features that are described in the context of a single embodiment can also be implemented in multiple embodiments separately or in any suitable subcombination. Moreover, although features may be described herein as acting in certain combinations and/or initially claimed as such, one or more features from a claimed combination can in some cases be excised from the combination, and the claimed combination may be directed to a subcombination or variation of a subcombination. Similarly, while operations may be described in a particular order, this should not be understood as requiring that such operations be performed in the particular order or in sequential order, or that all operations be performed, to achieve desirable results. Particular embodiments of the subject matter have been described. Other embodiments are within the scope of the following claims.

What is claimed is:

1. A method or system to use de-identified images collected from patients in association with de-identified data collected as part of medical care to train machine learning, machine vision, deep learning, or other algorithms that associate an outcome variable with an input image or video image(s).

2. A method or system to store de-identified images or video images for use in machine vision, machine learning, neural network, deep learning, or other algorithms that associate an outcome variable with an input image or video image(s).

3. A method or system to use unsupervised learning based on video images and data collected as part of medical care to train machine learning, machine vision, deep learning, or other algorithms that associated an outcome variable with an input image or video image(s).

4. A method or system to use unsupervised learning based on de-identified video images and de-identified data collected as part of medical care to train machine learning, machine vision, deep learning, or other algorithms that associated an outcome variable with an input image or video image(s).
